(19) United States
(12) Patent Application Publication
(10) Pub. No.: US 2017/0049832 A1
(43) Pub. Date: Feb. 23, 2017

(54) COMPOSITION COMPRISING DENDROPANAX MORBIFERA EXTRACT

(71) Applicant: Seung Hun LEE, Sedona, AZ (US)

(72) Inventor: Seung Hun LEE, Sedona, AZ (US)

(21) Appl. No.: 15/221,527

(22) Filed: Jul. 27, 2016

(30) Foreign Application Priority Data
Aug. 20, 2015 (KR) 1020150117387

(51) Int. Cl. A61K 36/25; A61Q 17/04; C11B 9/02; A61K 8/97

(52) U.S. Cl. CPC A61K 36/25; A61K 8/97; A61Q 17/04; C11B 9/025

(57) ABSTRACT

Disclosed is a composition containing a Dendropanax morbifera extract. The Dendropanax morbifera extract is used as an active ingredient for relieving stress, blocking UV light, and/or diffusing fragrance. The composition includes essential oil extracted from Dendropanax morbifera leaf, stem or sap, in which the Dendropanax morbifera extract is separated under optimal extraction conditions, which are determined by comparing and evaluating the components and yields of the essential oil, thus ensuring maximum efficacy and various applications of Dendropanax morbifera.

### BACKGROUND OF THE INVENTION

1. Technical Field

The present invention relates to a composition containing a Dendropanax morbifera extract and, more particularly, to a functional composition that includes a Dendropanax morbifera extract and thus has maximized efficacy, wherein the Dendropanax morbifera extract is separated under optimal extraction conditions, which are determined by comparing and evaluating the components and yields depending on the parts of Dendropanax morbifera and the extraction methods thereof.

2. Description of the Related Art

Typically, essential oils (extracts) obtained by extracting plant leaves or stems contain a variety of components (compounds). Plant essential oils are composed of terpene, which may be classified into, depending on the functional group thereof, alcohol, ketone, ether, ester, acid, and oxide. The essential oils of plants are composed mainly of monoterpene and include components such as sesquiterpene and diterpene in small amounts.

Various components that constitute the essential oils of plants are known to decrease blood cholesterol levels and to exhibit soothing effects, antibacterial effects and insecticidal effects. Accordingly, plant essential oils have been used for religious, medicinal and cosmetic applications for a long time in both the East and the West, and recently, essential oils have also come to be utilized in functional foods, functional cosmetics, aromatherapy products, etc., as well as the use of the unique fragrances thereof.

These days, mental stress of people may cause not only mental illnesses, such as anxiety and depression, but also diverse diseases, including tension headaches, migraines, pain, neurosis, hypertension, diabetes, indigestion, digestive ulcers, decrease in human physiological and immune activities, lowered body resistance, emotional instability, menopause-related disorders, decreased sexual function and so on. Furthermore, such mental stress is regarded as acting on the central nervous system, endocrine system and other life processes to thus cause typical disorders such as anxiety or depression.

In particular, plant essential oils are very effective when applied to the treatment of mental and physical disorders attributable to the aforementioned stress in modern people, and are thus receiving attention as alternative medicine in developed countries, and thorough research into the physiological activities thereof is ongoing.

For example, Korean Patent No. 10-0843680 discloses a fragrance composition for relieving stress, comprising a lavender essential oil, a lemon essential oil, a peppermint essential oil, a juniper berry essential oil, a marjoram essential oil, and a eucalyptus essential oil, and Korean Patent Application Publication No. 10-2012-0059207 discloses a pharmaceutical composition for reducing and treating insomnia containing a Cordyceps extract. Also, Korean Patent No. 10-1207781 discloses a composition for preventing and treating anxiety associated with cranial nerves and neurosis, containing, as an active ingredient, a compound separated from a Chrysanthemum indicum extract.

Dendropanax morbifera is known to grow wild only in the southwest coastal and island regions (Jeju Island, Wando, etc.) of Korea. Dendropanax morbifera has been mainly used as a natural golden paint for handicrafts in the past. Currently, Dendropanax morbifera, which grows wild in Korea, may generate many byproducts such as leaves or stems during the thinning and pruning processes.

Although Dendropanax morbifera is effective at improving various physiological effects and reducing mental illnesses, the usability thereof has not yet been reviewed. In particular, the leaves or stems of Dendropanax morbifera, generated during the thinning and pruning processes, are not appropriately utilized, but are mostly discarded.

### CITATION LIST

### Patent Literature

(Patent Document 1) Korean Patent No. 10-0843680

(Patent Document 2) Korean Patent Application Publication No. 10-2012-0059207

(Patent Document 3) Korean Patent No. 10-1207781

### SUMMARY OF THE INVENTION

Accordingly, the present invention has been made keeping in mind the problems encountered in the related art, and an object of the present invention is to provide a composition containing a Dendropanax morbifera extract as an active ingredient, in order to relieve stress (to reduce mental illness) based on the results of a study on the usability of Dendropanax morbifera.

Another object of the present invention is to provide a functional composition having maximized efficacy, which includes essential oil extracted from Dendropanax morbifera leaf, stem or sap, wherein the essential oil is separated under optimal extraction conditions, which are determined by comparing and evaluating the components and yields of the essential oil.

A first embodiment of the present invention provides a composition containing a Dendropanax morbifera extract as an active ingredient. A second embodiment of the present invention provides a composition containing a Dendropanax morbifera extract as an active ingredient for relieving stress, blocking UV light, and/or diffusing fragrance.

In an exemplary embodiment of the present invention, the Dendropanax morbifera extract may include at least one selected from a Dendropanax morbifera leaf extract, a Dendropanax morbifera stem (branch) extract, and a Dendropanax morbifera sap extract. The leaf extract may be a supercritical extract, and the stem extract may be a solvent extract. Furthermore, the sap extract may be a solvent fraction extract. The extract of each part of Dendropanax morbifera may include, for example, 5 wt % or more of terpene.

According to the present invention, the composition includes essential oil extracted from Dendropanax morbifera leaf, stem or sap, in which the Dendropanax morbifera extract is separated under optimal extraction conditions, which are determined by comparing and evaluating the components and yields of the essential oil, thus ensuring maximum efficacy and various applications of Dendropanax morbifera.

### BRIEF DESCRIPTION OF THE DRAWINGS

The above and other objects, features and advantages of the present invention will be more clearly understood from the following detailed description taken in conjunction with the accompanying drawings, in which:

FIG. 1 illustrates the sample photographs of the parts of Dendropanax morbifera;

FIG. 2 illustrates the steam distillation extractor used in an example of the present invention;

FIG. 3 illustrates the results of gas chromatography mass spectrometry (GC/MC) for the solvent extract of the Dendropanax morbifera leaf;

FIG. 4 illustrates the results of GC/MC for the solvent extract of the Dendropanax morbifera stem;

FIG. 5 illustrates the results of GC/MC for the supercritical extract of the Dendropanax morbifera leaf;

FIG. 6 illustrates the results of GC/MC for the supercritical extract of the Dendropanax morbifera stem; and

FIG. 7 illustrates the results of GC/MC for the solvent fraction extract of the Dendropanax morbifera sap.

### DESCRIPTION OF SPECIFIC EMBODIMENTS

As used herein, the term “and/or” includes any and all combinations of at least one of the associated listed items. As used herein, the term “at least one” is intended to include one or plural numbers of two or more. In the following description, a detailed description of the functions and/or constructions of conventional elements related with the present invention will be omitted.

The present invention addresses a composition containing a Dendropanax morbifera extract as an active ingredient. The composition according to the present invention is not limited as to its end use and dosage form, so long as it contains the Dendropanax morbifera extract.

In an exemplary embodiment, the composition according to the present invention may be selected from among a stress-relieving composition (a stress reliever), a UV-blocking composition (a UV blocker), and a fragrance-diffusing composition (a fragrance diffuser). For example, the composition according to the present invention may contain the Dendropanax morbifera extract as an active ingredient for relieving stress, blocking UV light, and/or diffusing fragrance. Specifically, in the composition according to the present invention containing the Dendropanax morbifera extract as the active ingredient, the Dendropanax morbifera extract may function as the active ingredient that is responsible for at least one function selected from among a stress relieving function, a UV blocking function, and a fragrance diffusing function. Also, the Dendropanax morbifera extract may act as an active ingredient for decreasing blood cholesterol levels and exhibiting soothing effects, antibacterial effects, and/or insecticidal effects.

In the present invention, stress relief results from alleviating mental illness, such as anxiety, depression and insomnia, and includes the meanings of prevention, reduction, and/or treatment of mental illness. Also, in the present invention, stress relief includes the meanings of prevention, reduction, and/or treatment of tension headaches, migraines, pain, neurosis, hypertension, diabetes, indigestion, digestive ulcers, decrease in human physiological and immune activities, lowered body resistance, emotional instability, menopause-related disorders, and/or decreased sexual function, all of which may result from mental illness.

Dendropanax morbifera is a broadleaf evergreen belonging to the family Araliaceae, and is known to both grow wild and be cultivated in Jeju Island and in the southwest coastal and island regions of Korea, including Wando, Bogildo Island, Eocheongdo Island, Jindo Island, Hongdo Island, Geomundo Island, and Yeonildo Island. Such Dendropanax morbifera contains aromatic components such as terpene and various components such as esters, ethers, acids, alcohols, and ketones.

The composition according to the present invention includes the extracts of Dendropanax morbifera leaf, stem (branch), sap, root and/or fruit. In the present invention, the Dendropanax morbifera extract includes the extract of at least one selected from among the leaf, stem (branch), sap, root, and fruit of Dendropanax morbifera, and is provided in a liquid phase, an oil phase, and/or a powder phase.

The Dendropanax morbifera extract may be obtained using various extraction methods, examples of which include solvent extraction using a solvent, supercritical extraction under supercritical conditions, steam distillation extraction using water vapor, and/or solvent fractionation extraction using an organic solvent. Each extraction method may be performed under typical conditions through various processes.

In the present invention, the Dendropanax morbifera extract may include not only the extract obtained through the above extraction method, but also the extract resulting from the purification process. Particularly, as the Dendropanax morbifera extract according to the present invention, a liquid extract obtained through the above extraction method may be used as it is, or the extract may be used after purification, for example, membrane separation using a separator such as an ultrafine filter membrane having a predetermined molecular weight cut-off value or any kind of chromatography (for separation according to size, electric charge, hydrophobicity or hydrophilicity). Moreover, in the composition of the present invention, the Dendropanax morbifera extract may be provided in the form of a concentrate resulting from concentrating the liquid extract, obtained through the above extraction method, under reduced pressure, a powder obtained through additional processing such as vacuum distillation and drying (lyophilization or spray drying), or a dilute solution obtained by diluting the powder in a solvent.

The solvent extraction may be performed once or at least two times in the temperature range from ambient temperature to 150° C. for 2 to 72 hr. In the present invention, ambient temperature may vary depending on the season, and typically ranges from −5 to 30° C. In the present invention, the solvent extraction includes not only extraction using an organic solvent but also hydrothermal extraction using water. Specifically, the extraction solvent used for the solvent extraction may include water and/or an organic solvent. The solvent extraction may be performed using at least one extraction solvent selected from among water and anhydrous or hydrous lower alcohols having 1 to 4 carbon atoms (methanol, ethanol, propanol, and butanol). Any solvent extract may be included in the present invention so long as it exhibits substantially the same effects even when it is obtained using extraction solvents, other than the above extraction solvent.

The supercritical extraction may be performed at a temperature of 25 to 60° C. under a pressure of 100 to 500 bar for 30 min to 5 hr. As such, the ratio of the flow rates of the CO₂ and the solvent, which are fed to the supercritical extractor, may range from 15 to 50:1 to 5, and the solvent may include, for example, ethyl alcohol.

The steam distillation extraction may be performed using a distillation extractor with a condenser through heating for 30 min to 5 hr using water vapor at 100° C. and cooling and capturing of the vaporized compounds.

The solvent fractionation extraction may be performed using an organic solvent such as hexane or ethyl acetate. The solvent fractionation extraction may be carried out in a manner in which the extract, obtained through solvent extraction, supercritical extraction and/or steam distillation extraction, is subjected to fractionation extraction using an organic solvent such as hexane.

Another aspect of the present invention addresses a method of preparing a Dendropanax morbifera extract suitable for the optimal end use depending on the extraction process of each part thereof. In the present invention, the components and yields are compared and evaluated depending on the parts of Dendropanax morbifera and the extraction methods thereof, and each part is extracted under optimal conditions to thus increase the active ingredient content. Thereby, a composition with maximized efficacy according to the end use thereof and a method for optimally extracting each part are provided.

In an exemplary embodiment of the present invention, the Dendropanax morbifera extract includes at least one selected from among a Dendropanax morbifera leaf extract, a Dendropanax morbifera stem (branch) extract, and a Dendropanax morbifera sap extract. As such, the extract of each part is obtained via solvent extraction, supercritical extraction, steam distillation extraction, and/or solvent fractionation extraction. Also, the sap extract may result from solvent fractionation extraction of the Dendropanax morbifera sap, but may be obtained by subjecting the Dendropanax morbifera sap to solvent extraction, supercritical extraction, and/or steam distillation extraction and then to solvent fractionation extraction.

According to a first embodiment of the present invention, the Dendropanax morbifera leaf extract is a solvent extract, and may include 30 to 40 wt % of propanoic acid ethyl ester, based on the total weight of the solvent extract. Also, the solvent extract of the Dendropanax morbifera leaf may include, based on the total weight of the solvent extract, about 8 to 14 wt % of cyclononasiloxane octadecamethyl and/or about 8 to wt % of tetracosamethyl-cyclododecasiloxane, and may additionally include oil, amine, terpene and/or an aromatic hydrocarbon (benzene, etc.) in an amount ranging from 0.1 to 40 wt %.

According to a second embodiment of the present invention, the Dendropanax morbifera leaf extract is a supercritical extract, and may include 5 to 40 wt % of terpene based on the total weight of the supercritical extract. As such, the terpene contained in the supercritical extract of the Dendropanax morbifera leaf may include alpha-amorphene and/or alpha-selinene. The supercritical extract of the Dendropanax morbifera leaf may include, based on the total weight of the supercritical extract, about 30 to 40 wt % of fonenol and/or 10 to 25 wt % of delta-cadinene, and may additionally include naphthalene, oil, amine, and/or ester in an amount ranging from 0.1 to 40 wt %.

According to a third embodiment of the present invention, the Dendropanax morbifera stem extract is a supercritical extract, and may include 50 to 60 wt % of propanoic acid ethyl ester based on the total weight of the supercritical extract. Also, the supercritical extract of the Dendropanax morbifera stem may include, based on the total weight of the supercritical extract, about 8 to 14 wt % of acetic acid propyl ester and/or about 8 to 14 wt % of trimethyl benzene, and may additionally include terpene and/or oxide in an amount ranging from 0.1 to 30 wt %.

According to a fourth embodiment of the present invention, the Dendropanax morbifera stem extract is a solvent extract, and may include 10 to 50 wt % of terpene based on the total weight of the solvent extract. As such, the terpene contained in the solvent extract of the Dendropanax morbifera stem may include limonene, alpha-pinene, beta-pinene, sabinene, and/or beta-myrcene. The solvent extract of the Dendropanax morbifera stem may include, based on the total weight of the solvent extract, about 15 to 25 wt % of menthol, 10 to 20 wt % of menthone, and/or about 8 to 14 wt % of cyclohexanone 5-methyl-2-(1-methylethyl), and may additionally include oil, amine, ester, and/or aromatic hydrocarbon in an amount ranging from 0.1 to 40 wt %.

According to a fifth embodiment of the present invention, the Dendropanax morbifera sap extract is a solvent fraction extract, and may include 10 to 20 wt % of terpene based on the total weight of the solvent fraction extract. As such, the terpene contained in the solvent fraction extract of the Dendropanax morbifera sap may include beta-myrcene and/or alpha-pinene. The solvent fraction extract of the Dendropanax morbifera sap may include 80 to 90 wt % of dihydrocarvyl acetate based on the total weight of the solvent fraction extract.

As mentioned above, in the present invention, the Dendropanax morbifera extract may include the main component and the amount (yield) varying depending on the part of Dendropanax morbifera and the extraction method thereof, and may be produced so as to have optimal efficacy suitable for each end use depending on the main component of each extract and the amount (yield) thereof. Specifically, depending on the main component of each extract and the amount (yield) thereof, for example, this extract may be applied to products for relieving stress, blocking UV light, or diffusing fragrance.

In the composition according to the present invention containing the Dendropanax morbifera extract as the active ingredient, the amount of the Dendropanax morbifera extract may be set to 0.01 wt % or more based on the total weight of the composition, depending on the end use and/or dosage form thereof. In the composition according to the present invention, for example, the Dendropanax morbifera extract may be appropriately used in an amount of 0.01 to 99.9 wt %, 0.1 to 60 wt %, or 0.2 to 30 wt %.

The composition according to the present invention may be provided in the form of a product by being selected from among a pharmaceutical composition, a food composition, and a cosmetic composition. Also, the composition of the invention may be provided in the form of a fragrance product, for example, a fragrance diffuser or an aroma therapeutic agent.

The composition of the invention may be provided in a dosage form such as a solid, a liquid or a cream, and may be particularly formulated into a suspension, a syrup, a cream, a spray, a tablet, a pill, a powder, a granule, and/or a capsule. The composition of the invention may include an excipient for preparing a formulation, for example, starch, calcium carbonate, sucrose, lactose and/or gelatin. The composition of the invention may further include, as examples of additives acceptable in the food, pharmaceutical and/or cosmetic areas, a filler, an extender, a binder, a humectant, a disintegrant, and/or a diluent.

In the present invention, the cosmetic composition is incorporated herein so long as it is applied to the skin or hair of human bodies. Specific examples of the cosmetic composition may include, depending on the kind of product, cosmetics for showing beauty or protecting skin, soap for beauty and cleanliness of the skin, a skin disease therapeutic agent for treating or preventing skin disease, shampoo or rinse for cleanliness of hair, a dye for dyeing hair, a hair loss inhibitor for preventing hair loss, and a hair restorer for promoting hair growth. This composition may be provided as at least one product selected from among cosmetics, soap, shampoo, rinse, a dye, a skin therapeutic agent (an eczema therapeutic agent, etc.), a hair loss inhibitor, and a hair restorer. In some cases, the cosmetic composition may be applied to oral products such as toothpaste or mouth cleaner.

A better understanding of the present invention may be obtained through the following examples which are set forth to illustrate, but are not to be construed to limit the scope of the present invention.

### EXAMPLES

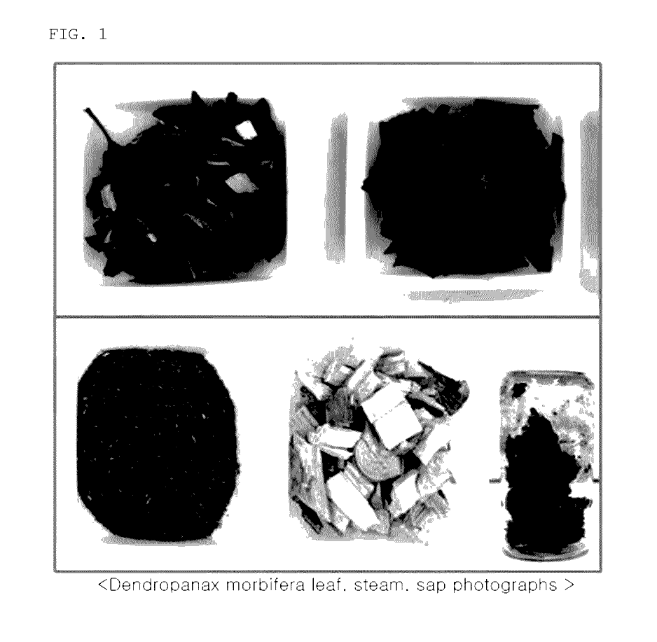

As part samples of Dendropanax morbifera, well-dried leaf, stem (branch) and sap of Dendropanax morbifera were prepared. FIG. 1 illustrates the sample photographs of the parts of Dendropanax morbifera. The crushed mixture sample of leaf and stem (top left of FIG. 1), the crushed sample of leaf (top right of FIG. 1), the ground sample of leaf (bottom left of FIG. 1), the crushed sample of stem (bottom middle of FIG. 1) and the solid sample of sap (bottom right of FIG. 1) were prepared, and then extracted as follows.

### Extract Preparation Example 1

### Solvent Extraction of Leaf, Stem and Sap

Each of the crushed sample of leaf, the crushed sample of stem (branch) and the solid sample of sap was subjected to solvent extraction using 70 wt % of ethanol (EtOH). Ethanol (EtOH) was added in an amount corresponding to 20 times the weight of each sample, and extraction was repeated two times at ambient temperature for 24 hr. Therefore, the resulting extract was allowed to stand for one day at ambient temperature and then filtered with filter paper (Whatman NO. 1, Whatman International Ltd., Maidstone, England). Subsequently, the filtered product was concentrated at 55° C. using a rotary evaporator (UT-1000, EYELA, Tokyo, Japan) and then lyophilized.

Extract Preparation Example 2:

Supercritical Extraction of Leaf and Stem

Each of the ground sample of leaf and the crushed sample of stem (branch) was subjected to supercritical extraction under the conditions shown in Table 1 below, thus obtaining a leaf extract and a stem extract.

TABLE 1
<Supercritical extraction conditions of leaf and
stem>

| | |
|---|---|
| Manufacturer and Model of Extractor | Waters, AD-RC08 |
| CO₂ flow rate: solvent flow rate | 47.5 mL/min: 2.5 mL/min |
| Total flow rate | Total flow rate: 50 mL/min |
| (CO₂ flow rate + solvent flow rate) | |
| Co-solvent | Ethyl alcohol (Purity: 96 wt %) |
| Temperature | 50° C. |
| Pressure | 400 bar |
| Extraction time | 120 min |

### Extract Preparation Example 3

### Steam Distillation Extraction of Leaf and Stem

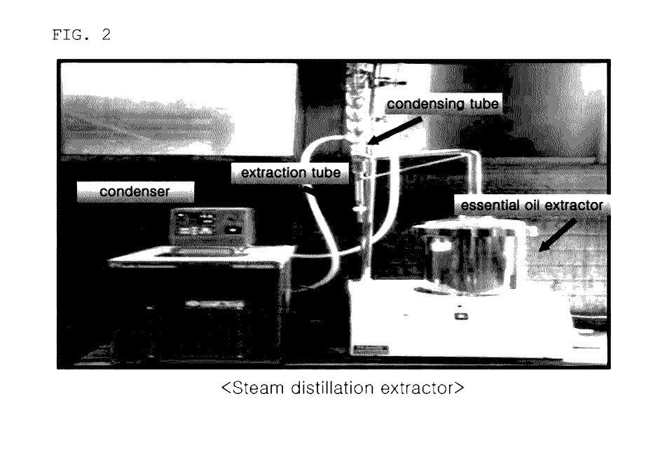

Each of the crushed sample of leaf and the crushed sample of stem (branch) was extracted using a steam distillation extractor. FIG. 2 illustrates the steam distillation extractor used for this process. As such, each sample was placed in a piece of nonwoven fabric, which was then placed in an essential oil extractor and heated for 120 min using water vapor at about 100° C., followed by cooling and capturing the vaporized compounds.

<Gas Chromatography Mass Spectrometry (GC/MS)>

Respective extracts of the parts (leaf, steam and sap) of Dendropanax morbifera depending on the extraction method (supercritical extraction, solvent extraction and steam distillation extraction) were derivatized with BSTFA and analyzed via GC/MS. Specifically, each extract was subjected to solvent fractionation using hexane [hexane/D.W (1:1)] to remove polar material therefrom. The obtained hexane fraction was diluted to 300 ppm, and the component and amount of each extract were qualitatively and quantitatively analyzed via GC/MS. As such, the top 20 components among the components of each extract were selected and analyzed using Wiley 8N Library program. The analytical device and conditions were as follows.

GC/MS-QP2010 Plus (Shimazu)

Column: DB-5 (0.25 mm×30 m, 0.25 um)

Column oven temp.: initially set at 40° C., held for 10 min, then ramped up to 220° C. at a rate of 4° C./rain and held for 5 min

Injection temp. (° C.): 280

Column flow: 1.09 mL/min.

Split ratio: 10

MS Condition

Ion source temp. (° C.): 250

Interface temp. (° C.): 280

Mass range: 60-600

<Analytical Results>

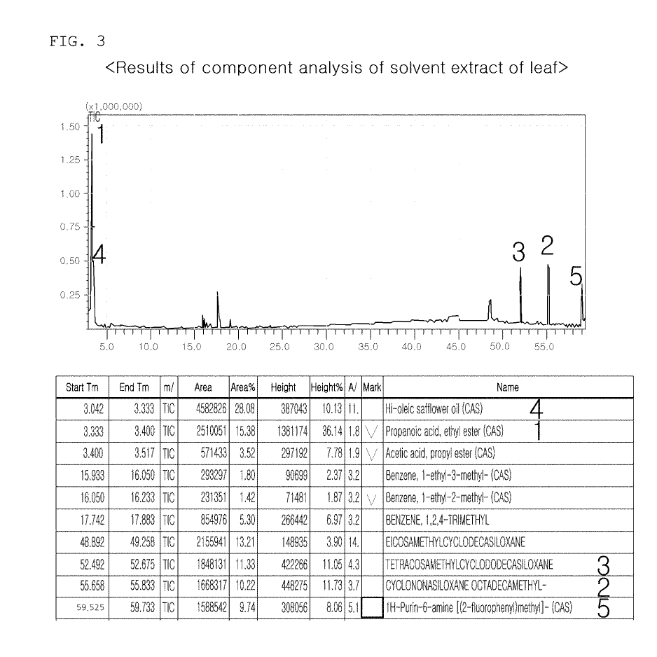

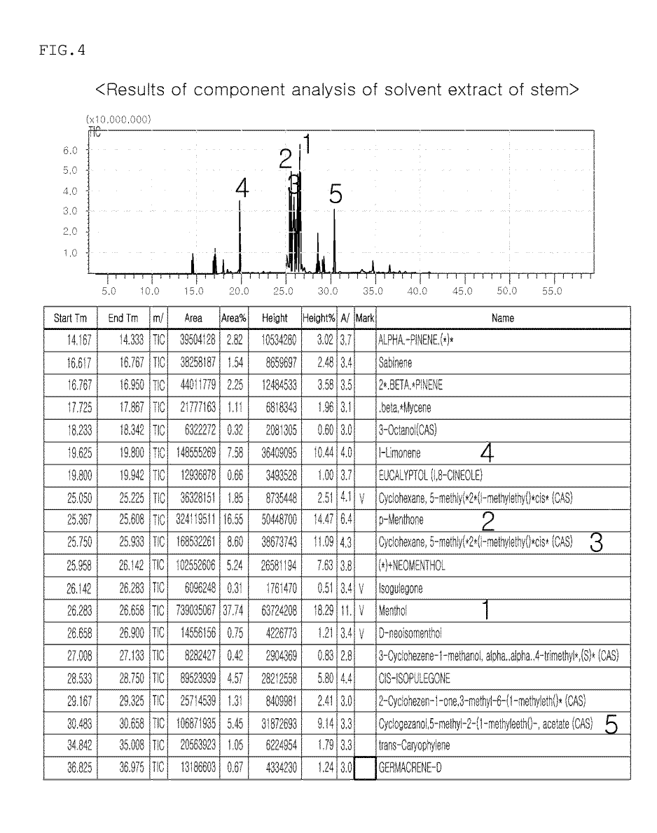

As the analytical results of the solvent extraction of the Dendropanax morbifera leaf and stem, FIG. 3 illustrates the results of analysis of the components of the solvent extract of the leaf, and FIG. 4 illustrates the results of analysis of the components of the solvent extract of the stem.

As illustrated in FIG. 3, in the solvent extract of the leaf, propanoic acid ethyl ester, showing a pungent odor (rancid odor), was detected in the largest amount of about 36.14 wt %, and cyclononasiloxane octadecamethyl, found in lettuce and the like, was detected in an amount of about 11.73 wt %, and tetracosamethyl-cyclododecasiloxane was detected in an amount of 11.05 wt %. Further, hi-oleic safflower oil, 2-fluorophenyl methyl-1H-purine-6-amine, acetic acid, and benzene, in that order, were detected.

As illustrated in FIG. 4, in the solvent extract of the stem, menthol was detected in the largest amount, of about 18.29 wt %, followed by about 14.47 wt % of p-menthone, about 11.09 wt % of cyclohexanone 5-methyl-2-(1-methylethyl), about 10.44 wt % of 1-limonene, and about 9.14 wt % of cyclohexanone 5-methyl-2-(1-methylethyl)-acetate. Furthermore, as the monoterpene of the well-known aromatic essential oil, alpha-pinene, sabinene and beta-myrcene were detected.

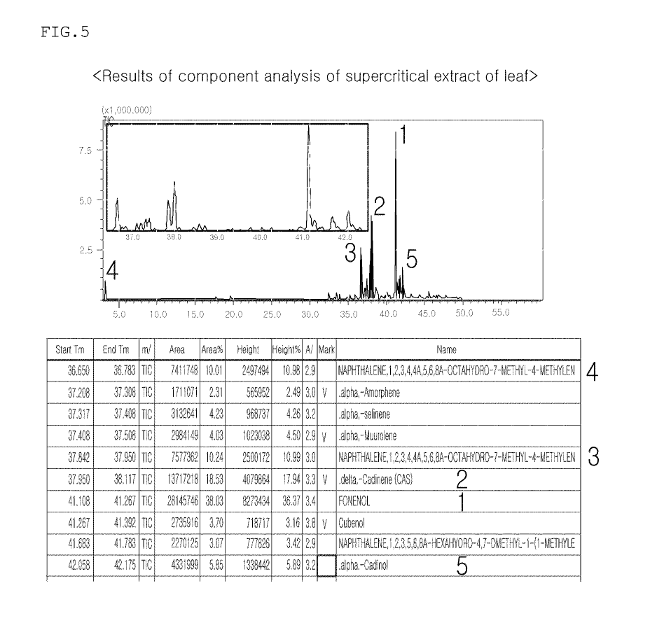

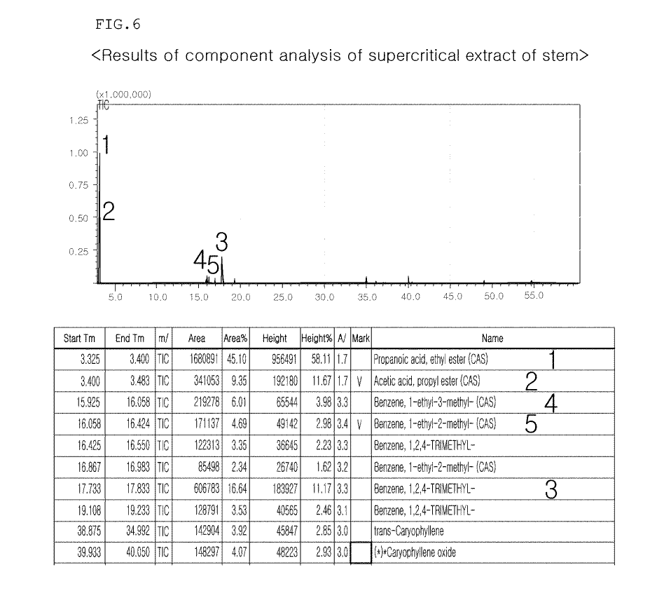

FIGS. 5 and 6 illustrate the analytical results of the supercritical extraction of the Dendropanax morbifera leaf and stem. FIG. 5 shows the results of analysis of the components of the supercritical extract of the leaf, and FIG. 6 shows the results of analysis of the components of the supercritical extract of the stem.

As shown in FIG. 5, in the supercritical extract of the leaf, fonenol was detected in the largest amount of about 36.37 wt %, after which delta-cadinene was detected in an amount of about 17.94 wt %. Furthermore, alpha-amorphene and alpha-selinene were detected, and naphthalene, known as the main component of the stem of the Acanthopanax tree, was detected.

As shown in FIG. 6, in the supercritical extract of the stem, propanoic acid ethyl ester, showing a pungent odor (rancid odor) was mainly detected in an amount of about 58.11 wt %, and then about 11.67 wt % of acetic acid propyl ester and about 11.17 wt % of trimethyl benzene were detected.

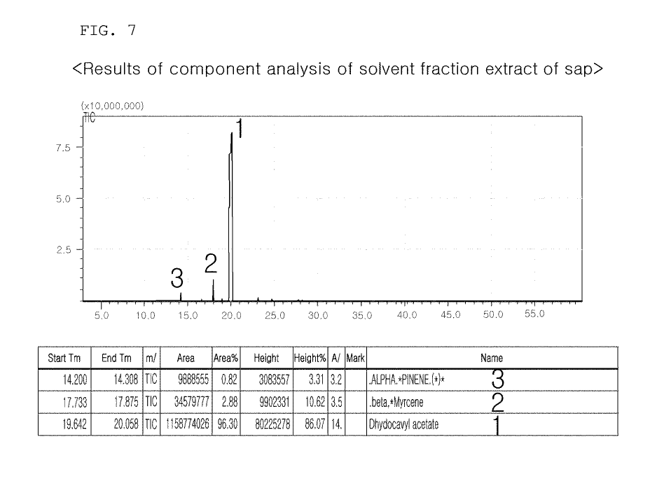

FIG. 7 illustrates the results of analysis of the components of the solvent (hexane) fraction extract of the Dendropanax morbifera sap.

As illustrated in FIG. 7, in the solvent (hexane) fraction extract of the sap, dihydrocarvyl acetate, which is the fruity, minty fragrance component of fresh herbs, was mainly detected in an amount of about 86.07 wt %, and furthermore, about 10.52 wt % of beta-myrcene and about 3.31 wt % of alpha-pinene were detected.

Based on the above results, depending on the part of Dendropanax morbifera and the extraction method thereof, the kind of main component and the amount thereof became different. The extraction method that is suitable for each part may be selected appropriately, and the kind and amount (yield) of the active ingredient may be optimized so as to be adapted for the end use thereof.

As mentioned above, the Dendropanax morbifera leaf contained about 36.14 wt % of propanoic acid ethyl ester and about 11.73 wt % of cyclononasiloxane octadecamethyl when subjected to solvent extraction, and the supercritical extract thereof was composed of about 36.37 wt % of fonenol and about 17.94 wt % of delta-cadinene. The Dendropanax morbifera stem (branch) contained about 18.29 wt % of menthol and about 14.47 wt % of p-menthone when subjected to solvent extraction, and the supercritical extract thereof was composed of about 58.11 wt % of propanoic acid ethyl ester and about 11.67 wt % of acetic acid propyl ester. The solvent fraction of the Dendropanax morbifera sap contained about 86.07 wt % of dihydrocarvyl acetate.

As for kinds of terpene that are well-known as aromatic essential oil components, alpha-amorphene and alpha-selinene were detected in the supercritical extract of the leaf, and alpha-pinene, sabinene, beta-myrcene and 1-limonene were detected in the solvent extract of the stem. Also, beta-myrcene and alpha-pinene were detected in the solvent fraction of the sap.

Therefore, when the Dendropanax morbifera extract is used for diffusing fragrance (aroma therapy, etc.), the use of the supercritical extract for the leaf and the solvent extract for the stem can be found to be optimal.

### Formulation Example 1

### UV Blocker

The Dendropanax morbifera leaf and stem, cut to a predetermined size, were mixed at a weight ratio of 2:1, immersed in an alum aqueous solution at ambient temperature for 2 hr, and then dried with sunlight. The dried product and purified water were mixed at a weight ratio of 1:4 and then subjected to warm extraction at 80° C. for 12 hr.

Next, the warm extract was cooled, impurities were removed therefrom, and the extract was concentrated under reduced pressure and was then lyophilized, thus obtaining a mixed extract (powder) for a UV blocker. Also, a UV blocking base was prepared using components in the amounts shown in Table 2 below, and was then mixed with the above mixed extract obtained through warm extraction.

TABLE 2
<Composition for UV blocker>

| Component | Amount (wt %) |
|---|---|
| Purified water | 69.5 |
| Octocrylene | 10.0 |
| Glycerin | 6.0 |
| Ethylhexyl salicylate | 4.5 |
| C12-15 Alkylbenzoate | 3.0 |
| Titanium oxide | 2.0 |
| Aluminum oxide | 2.0 |
| Zinc oxide | 2.0 |
| Phenoxyethanol | 0.5 |
| Sorbitan stearate | 0.2 |
| Disodium EDTA | 0.1 |
| Xanthan gum | 0.1 |
| Dendropanax morbifera extract | 0.1 |
| Total | 100.0 |

### Formulation Example 2

### Fragrance Diffuser (Stress Reliever)

An oil mixture comprising jojoba oil and apricot seed oil was added with the sap extract and the stem extract, obtained through solvent extraction in Extract Preparation Example 1, and the resulting mixture was used as a healing fragrance diffuser (a stress reliever). As shown in Table 3 below, the mixing ratio of jojoba oil and apricot seed oil and the amount of each extract that was used were changed in Examples. In Table 3 below, Comparative Example was composed exclusively of the oil mixture comprising jojoba oil and apricot seed oil, without any Dendropanax morbifera extract.

TABLE 3
<Fragrance diffuser composition, wt %>

| Component | Ex. 1 | Ex. 2 | Comp. Ex. |
|---|---|---|---|
| Jojoba oil | 30.0 | 20.0 | 50.0 |
| Apricot seed oil | 30.0 | 40.0 | 50.0 |
| Dendropanax morbifera sap | 30.0 | 20.0 | — |
| extract | | | |
| Dendropanax morbifera stem | 10.0 | 20.0 | — |
| extract | | | |

The fragrance diffusers of Examples 1 and 2 and Comparative Example were applied to subjects for 6 hr/day over a total of 30 days to evaluate the extent of reduction of insomnia. The subjects were 20 male adults, ranging in age from 40 to 50 and suffering from insomnia due to stress. The evaluation was based on the following criteria. The results are shown in Table 4 below.

<Evaluation Criteria>

Point 1: No reduction

Point 2: Slight reduction

Point 3: Significant reduction

TABLE 4
<Evaluation results for reduction of insomnia>

| No. | Ex. 1 | Ex. 2 | Comp. Ex. |
|---|---|---|---|
| Reduction (average) | 2.2 | 2.6 | 1.8 |

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition comprising a Dendropanax morbifera extract as an active ingredient.

2. A composition comprising a Dendropanax morbifera extract as an active ingredient for relieving stress, blocking UV light or diffusing a fragrance.

3. The composition of claim 1, wherein the Dendropanax morbifera extract comprises at least one selected from among a leaf extract, a stem extract, and a sap extract.

4. The composition of claim 3, wherein the leaf extract is a supercritical extract, and the stem extract is a solvent extract.

5. The composition of claim 3, wherein the leaf extract is a solvent extract, and includes 30 to 40 wt % of propanoic acid ethyl ester based on a total weight of the solvent extract, and the stem extract is a supercritical extract, and includes 50 to 60 wt % of propanoic acid ethyl ester based on a total weight of the supercritical extract.

6. The composition of claim 3, wherein the leaf extract is a supercritical extract, and includes 5 to 40 wt % of terpene based on a total weight of the supercritical extract.

7. The composition of claim 3, wherein the stem extract is a solvent extract, and includes 10 to 50 wt % of terpene based on a total weight of the solvent extract.

8. The composition of claim 3, wherein the sap extract is a solvent fraction extract, and includes 10 to 20 wt % of terpene based on a total weight of the solvent fraction extract.

9. The composition of claim 2, wherein the Dendropanax morbifera extract comprises at least one selected from among a leaf extract, a stem extract, and a sap extract.

10. The composition of claim 9, wherein the leaf extract is a solvent extract, and includes 30 to 40 wt % of propanoic acid ethyl ester based on a total weight of the solvent extract, and the stem extract is a supercritical extract, and includes 50 to 60 wt % of propanoic acid ethyl ester based on a total weight of the supercritical extract.

11. The composition of claim 9, wherein the leaf extract is a supercritical extract, and includes 5 to 40 wt % of terpene based on a total weight of the supercritical extract.

12. The composition of claim 9, wherein the stem extract is a solvent extract, and includes 10 to 50 wt % of terpene based on a total weight of the solvent extract.

13. The composition of claim 9, wherein the sap extract is a solvent fraction extract, and includes 10 to 20 wt % of terpene based on a total weight of the solvent fraction extract.
